(19) United States
(12) Patent Application Publication
(10) Pub. No.: US 2025/0340681 A1
(43) Pub. Date: Nov. 6, 2025

(54) RADIOPAQUE POLYMER FORMULATIONS COMPRISING THERMOPLASTIC POLYMER, TUNGSTEN, AND BARIUM SULFATE

(71) Applicant: Avient Corporation, Avon Lake, OH (US)

(72) Inventor: Karan PATEL, Karlsruhe (DE)

(73) Assignee: Avient Corporation, Avon Lake, OH (US)

(21) Appl. No.: 18/854,060

(22) PCT Filed: Apr. 4, 2023
(86) PCT No.: PCT/US2023/017431

§ 371 Date: Oct. 4, 2024

Related U.S. Application Data
(60) Provisional application No. 63/328,489, filed on Apr. 7, 2022.

(51) Int. Cl. C08F 10/06; C08F 10/02; C08K 3/08; C08K 3/30; C08K 5/20; C08K 5/372

(52) U.S. Cl. CPC C08F 10/06; C08F 10/02; C08K 3/08; C08K 3/30; C08K 5/20; C08K 5/372; C08K 2003/0887; C08K 2003/3045

(57) ABSTRACT

Embodiments of the present disclosure are directed to radiopaque polymer formulations comprising thermoplastic polymer and a filler. The filler comprises 25 wt % to 45 wt % of tungsten and 35 wt % to 55 wt % of barium sulfate.

### CLAIM OF PRIORITY

This application claims the benefit of U.S. Provisional Patent Application Ser. No. 63/328,489 bearing Attorney Docket Number 1202205 and filed on Apr. 7, 2022, which is hereby incorporated by reference in its entirety.

### TECHNICAL FIELD

Embodiments of the present disclosure are generally related to radiopaque polymer formulations of thermoplastic polymer and a filler comprising tungsten and barium sulfate.

### BACKGROUND

Polymers used to produce catheters and other medical devices that are inserted into the body for diagnostic or interventional procedures are commonly filled with substances opaque to x-rays, thereby rendering the devices visible under fluoroscopy or x-ray imaging. Tungsten and barium sulfate are widely used as fillers for medical devices. However, these fillers may not provide the dielectric strength, the absorption, and the relatively low reflectivity desired for medical applications.

Accordingly, a continual need exists for fillers of radiopaque polymer formulations that provide desired dielectric strength, absorption, and reflectivity without degrading the mechanical properties, e.g., tensile strength, tensile elongation, flexural modulus, charpy impact strength, izod impact strength, melt flow rate, or combinations thereof, of the base polymers.

### SUMMARY

Embodiments of the present disclosure are directed to radiopaque polymer formulations, which mitigate the aforementioned problems. Specifically, the radiopaque polymer formulations disclosed herein comprise thermoplastic polymer and a filler comprising tungsten and barium sulfate, which result in radiopaque polymer formulations having a desired dielectric strength before x-ray exposure, after x-ray exposure, or both and desired absorption and reflectivity while maintaining the mechanical properties of the thermoplastic polymer.

According to one embodiment, a radiopaque polymer formulation is provided. The radiopaque polymer formulations comprises thermoplastic polymer and a filler. The filler comprises 25 weight percent (wt %) to 45 wt % of tungsten and 35 wt % to 55 wt % of barium sulfate, based on the total amount of the radiopaque polymer formulation.

According to one embodiment, a medical device is provided comprising a radioactive polymer that comprises thermoplastic polymer and a filler. The filler comprises 25 weight percent (wt %) to 45 wt % of tungsten and 35 wt % to 55 wt % of barium sulfate, based on the total amount of the radiopaque polymer formulation. In embodiments, the medical device may be an X-ray shielding component.

Additional features and advantages of the embodiments described herein will be set forth in the detailed description which follows, and in part will be readily apparent to those skilled in the art from that description or recognized by practicing the embodiments described herein, including the detailed description, which follows and the claims.

### DETAILED DESCRIPTION

Reference will now be made in detail to various embodiments of formulations, specifically radiopaque polymer formulations comprising thermoplastic polymer and a filler. The filler comprises 25 wt % to 45 wt % of tungsten and 35 wt % to 55 wt % of barium sulfate, based on the total amount of the radiopaque polymer formulation.

The disclosure should not be construed as limited to the embodiments set forth herein. Rather, these embodiments are provided so that this disclosure will be thorough and complete, and will fully convey the subject matter to those skilled in the art.

### Definitions

Unless otherwise defined, all technical and scientific terms used herein have the same meaning as commonly understood by one of ordinary skill in the art. The terminology used in the disclosure herein is for describing particular embodiments only and is not intended to be limiting.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used in the specification and the appended claims, the singular forms “a,” “an,” and “the” are intended to include the plural forms as well, unless the context clearly indicates otherwise. Thus, for example, reference to “a” component includes aspects having two or more such components, unless the context clearly indicates otherwise.

The terms “consist essentially of” or “consisting essentially of,” as described herein, limits the scope of a material to the specified material compositions and those that do not materially affect the basic characteristics of the material.

The terms “weight percent” or “wt %,” as described herein, refers to the weight fraction of the component of the radiopaque polymer formulation based on the total amount (i.e., weight) of the radiopaque polymer formulation, unless otherwise noted.

The term “charpy impact strength,” as described herein, refers to a toughness of a material measured by a charpy impact test according to ISO 179-1:2010.

The term “izod impact strength,” as described herein, refers to a strength of a material measured by an izod impact test according to ISO 180:2019.

The term “tensile strength at break,” as described herein, refers to the maximum stress that a material can withstand while stretching before breaking as measured according to ISO 527-2:2012 at 23° C. and a rate of strain of 5 mm/min.

The term “tensile elongation at break,” as described herein, refers to the ratio between increased length and initial length after breakage as measured according to ISO 527-2:2012 at 23° C. and a rate of strain of 5 mm/min.

The term “flexural modulus,” as described herein, refers the ratio of stress to strain in flexural deformation as measured according to ISO 178:2019 at 23° C. and a rate of strain 2 mm/min.

The term “melt flow rate,” as described herein, refers to the ability of a material's melt to flow under pressure as measured according to ASTM D1238 or ISO 1133-2:2011 at 230° C. and 5 kg.

The term “dielectric strength” or “breakdown strength,” as described herein, refers to the minimum voltage for spark breakdown to occur across a material held between electrodes producing a uniform electric field as measured according to VDE 0370/Teil5/96.

The term “breakdown strength after X-ray exposure,” as described herein, refers to the minimum voltage for spark breakdown to occur across a material held between electrodes producing a uniform electric field after the material is exposed to X-ray as measured according to VDE 0370/Teil5/96 after the material is exposed at 900 kGy (kilogray).

The term “absorption of 1 mm Pb,” as described herein, refers to a measure of the quantity of light of 1 mm Pb absorbed by a material as measured using Smiths Heimann Detection Hi-Ray 10X.

The term “density,” as described herein, refers to the mass per unit volume of a substance as measured according to ISO 1183-1:2019 at 23° C.

The term “copolymer,” as described herein, refers to a polymer formed when two or more types or species of monomers are linked in the same chain.

The term “radiopaque,” as described herein, refers to the ability of a substance to block x-rays (for example through absorption) with wavelengths ranging from about 10⁻⁸ to 10⁻¹² meter to an extent that the substance is visible under fluoroscopy or x-ray imaging. In contrast, non-radiopaque substances allow for the x-rays to pass through the substance or are only partially blocked the substance to a small extent.

The term “thermoplastic polymer,” as described herein, refers to a polymer material that is capable of becoming pliable or moldable when the temperature is raised above a certain temperature and solidifies upon cooling below the certain temperature.

The term “thermoplastic polyolefin,” as described herein, refers to a polyolefin material that is capable of becoming pliable or moldable when the temperature is raised above a certain temperature and solidifies upon cooling below the certain temperature.

The term “thermoplastic elastomer,” as described herein, refers an elastomer material that is capable of becoming pliable or moldable when the temperature is raised above a certain temperature and solidifies upon cooling below the certain temperature.

The terms “reflectively” or “reflectance” as described herein, refers to an ability of a material to reflect the energy incident on its surface.

The term “low reflectivity,” as described herein, refers to a reflectance lower than the reflectance of tungsten.

The term “high reflectivity,” as described herein, refers to the reflectance of tungsten.

The term “high dielectric strength,” as described herein, refers to a dielectric strength under DC load greater than or equal to 3 kV/mm.

The term “high absorption,” as described herein, refers to an absorption of 1 mm lead (Pb) greater than or equal to 5 mm.

Polymers used to produce catheters and other medical devices that are inserted into the body for diagnostic or interventional procedures are commonly filled with substances opaque to x-rays, thereby rendering the devices visible under fluoroscopy or x-ray imaging. These fillers affect the energy attenuation of photons in an x-ray beam as it passes through matter, reducing the intensity of the photons by absorbing or deflecting them. Image contrast and sharpness may be varied by the type and amount of fillers used, and may be tailored to the specific application of the medical devices.

As newer x-ray machines operate at higher energy levels than older ones, higher dielectric strength is required to provide sufficient stability after x-ray exposure while maintaining high absorption. Tungsten and barium sulfate are widely used as fillers for medical devices, but these fillers may not provide the dielectric strength, the absorption, and the relatively low reflectivity desired for medical applications. For example, tungsten has high dielectric strength, but has relatively high reflectivity. Barium sulfate has high dielectric strength and high absorption, but may not have the level of reflectivity required for x-ray applications. Further, increasing the amount of the filler may degrade the mechanical properties of the base polymer.

Disclosed herein are radiopaque polymer formulations which mitigate the aforementioned problems. Specifically, the radiopaque polymer formulations disclosed herein comprise thermoplastic polymer and a filler comprising tungsten and barium sulfate, which result in radiopaque polymer formulation having both desired dielectric strength before x-ray exposure, after x-ray exposure, or both and high absorption and low reflectivity that may be desired in medical applications, while maintaining the mechanical properties, such as tensile strength, tensile elongation, flexural modulus, charpy impact strength, izod impact strength, melt flow rate, or combinations thereof, of the thermoplastic polymer. The thermoplastic polymer imparts the desired mechanical properties (e.g., tensile strength, tensile elongation, flexural modulus, charpy impact strength, izod impact strength, melt flow rate, or combinations thereof). Tungsten provides the relatively high dielectric strength and reflectivity necessary for x-ray applications. Barium sulfate provides the relatively high dielectric strength and high absorption that may be desired in medical applications, such as X-ray devices.

The radiopaque polymer formulations disclosed herein may generally be described as comprising thermoplastic polymer and a filler.

### Thermoplastic Polymer

As described hereinabove, the presence and specific amount of thermoplastic polymer, along with a filler, produces a radiopaque polymer formulation having desired dielectric strength before x-ray exposure, after x-ray exposure, or both as well as high absorption and low reflectivity without degrading the mechanical properties, i.e. tensile strength, tensile elongation, flexural modulus, charpy impact strength, izod impact strength, melt flow rate, or combinations thereof, of base polymers. In particular, the thermoplastic polymer imparts the desired mechanical properties to the radiopaque polymer formulation. The thermoplastic polymer may be base polymers of the radiopaque polymer formulation.

Various thermoplastic polymers are considered suitable for the present radiopaque polymer formulation. In embodiments, the thermoplastic polymer may comprise thermoplastic polyolefin, thermoplastic elastomer, or both.

In embodiments, the thermoplastic polymer may comprise polyethylene, polypropylene (PP), polyphenylene sulfide (PPS), polybutylene terephthalate (PBT), thermoplastic polyester elastomers (TPC), polycarbonate, Acrylonitrile butadiene styrene (ABS) or a combination thereof.

In embodiments, the polypropylene may comprise a polypropylene homopolymer (i.e., composed of propylene monomers) or a polypropylene copolymer having greater than 50 wt % propylene monomer and an additional comonomer such as C₃-C₁₂ alpha olefins.

In embodiments, the polypropylene may comprise maleic anhydride modified homo polypropylene.

In embodiments, the polyethylene may comprise a polyethylene homopolymer (i.e., composed of ethylene monomers) or a polyethylene copolymer having greater than 50 wt % ethylene monomer and an additional comonomer, such as C₃-C₁₂ alpha olefins.

In embodiments, the amount of thermoplastic polymer in the radiopaque polymer formulation may be greater than 7 wt %, greater than or equal to 8 wt %, greater than or equal to 9 wt %, greater than or equal to 10 wt %, or even greater than or equal to 11 wt %. In embodiments, the amount of thermoplastic polymer in the radiopaque polymer formulation may be less than or equal to 35 wt %, less than or equal to 34 wt %, less than or equal to 33 wt %, less than or equal to 32 wt %, less than or equal to 31 wt %, or even less than or equal to 30 wt %. In embodiments, the amount of thermoplastic polymer in the radiopaque polymer formulation may be from 7 wt % to 35 wt %, from 7 wt % to 34 wt %, from 7 wt % to 33 wt %, from 7 wt % to 32 wt %, from 7 wt % to 31 wt %, from 7 wt % to 30 wt %, from 8 wt % to 35 wt %, from 8 wt % to 34 wt %, from 8 wt % to 33 wt %, from 8 wt % to 32 wt %, from 8 wt % to 31 wt %, from 8 wt % to 30 wt %, from 9 wt % to 35 wt %, from 9 wt % to 34 wt %, from 9 wt % to 33 wt %, from 9 wt % to 32 wt %, from 9 wt % to 31 wt %, from 9 wt % to 30 wt %, from 10 wt % to 35 wt %, from 10 wt % to 34 wt %, from 10 wt % to 33 wt %, from 10 wt % to 32 wt %, from 10 wt % to 31 wt %, from 10 wt % to 30 wt %, from 11 wt % to 35 wt %, from 11 wt % to 34 wt %, from 11 wt % to 33 wt %, from 11 wt % to 32 wt %, from 11 wt % to 31 wt %, or even from 11 wt % to 30 wt %, or any and all sub-ranges formed from any of these endpoints.

In embodiments, the thermoplastic polymer may comprise a density greater than or equal to 0.7 g/cm³ or even greater than or equal to 0.8 g/cm³. In embodiments, the thermoplastic polymer may comprise a density less than or equal to 1.3 g/cm³ or even less than or equal to 1.2 g/cm³. In embodiments, the thermoplastic polymer may comprise a density from 0.7 g/cm³ to 1.3 g/cm³, from 0.7 g/cm³ to 1.2 g/cm³, from 0.8 g/cm³ to 1.3 g/cm³, or even from 0.8 g/cm³ to 1.2 g/cm³, or any and all sub-ranges formed from any of these endpoints.

In embodiments, the thermoplastic polymer may comprise a melting point greater than or equal to 110° C. or even greater than or equal to 120° C. In embodiments, the thermoplastic polymer may comprise a melting point less than or equal to 190° C. or even less than or equal to 180° C. In embodiments, the thermoplastic polymer may comprise a melting point from 110° C. to 190° C., from 110° C. to 180° C., from 120° C. to 190° C., or even from 120° C. to 180° C., or any and all sub-ranges formed from any of these endpoints.

In embodiments, the thermoplastic polymer may have a tensile elongation at break greater than or equal to 600 percentage (%), greater than or equal to 650%, or even greater than or equal to 700%. In embodiments, the thermoplastic polymer may have a tensile elongation at break less than or equal to 1000%, less than or equal to 950%, or even less than or equal to 900%. In embodiments, the thermoplastic polymer may have a tensile elongation at break from 600% to 1000%, from 600% to 950%, from 600% to 900%, from 650% to 1000%, from 650% to 950%, from 650% to 900%, from 700% to 1000%, from 700% to 950%, or even from 700% to 900%, or any and all sub-ranges formed from any of these endpoints.

In embodiments, the thermoplastic polymer may have a tensile strength at break greater than or equal to 10 megapascal (mPa), greater than or equal to 15 mPa, or even greater than or equal to 20 mPa. In embodiments, the thermoplastic polymer may have a tensile strength at break less than or equal to 80 mPa, less than or equal to 70 mPa, or even less than or equal to 60 mPa. In embodiments, the thermoplastic polymer may have a tensile strength at break from 10 mPa to 80 mPa, from 10 mPa to 70 mPa, from 10 mPa to 60 mPa, from 15 mPa to 80 mPa, from 15 mPa to 70 mPa, from 15 mPa to 60 mPa, from 20 mPa to 80 mPa, from 20 mPa to 70 mPa, or even from 20 mPa to 60 mPa, or any and all sub-ranges formed from any of these endpoints.

In embodiments, the thermoplastic polymer may have a flexural modulus greater than or equal to 1000 mPa, greater than or equal to 1100 mPa, or even greater than or equal to 1200 mPa. In embodiments, the thermoplastic polymer may have a flexural modulus less than or equal to 1700 mPa, less than or equal to 1600 mPa, or even less than or equal to 1500 mPa. In embodiments, the thermoplastic polymer may have a flexural modulus from 1000 mPa to 1700 mPa, from 1000 mPa to 1600 mPa, from 1000 mPa to 1500 mPa, from 1100 mPa to 1700 mPa, from 1100 mPa to 1600 mPa, from 1100 mPa to 1500 mPa, from 1200 mPa to 1700 mPa, from 1200 mPa to 1600 mPa, or even from 1200 mPa to 1500 mPa, or any and all sub-ranges formed from any of these endpoints.

In embodiments, the thermoplastic polymer may have a melt flow rate greater than or equal to 30 g/10 min, greater than or equal to 35 g/10 min, or even greater than or equal to 40 g/10 min. In embodiments, the thermoplastic polymer may have a melt flow rate less than or equal to 90 g/10 min, less than or equal to 85 g/10 min, or even less than or equal to 80 g/10 min. In embodiments, the thermoplastic polymer may have a melt flow rate from 30 g/10 min to 90 g/10 min, from 30 g/10 min to 85 g/10 min, from 30 g/10 min to 80 g/10 min, from 35 g/10 min to 90 g/10 min, from 35 g/10 min to 85 g/10 min, from 35 g/10 min to 80 g/10 min, from 40 g/10 min to 90 g/10 min, from 40 g/10 min to 85 g/10 min, or even from 40 g/10 min to 80 g/10 min, or any and all sub-ranges formed from any of these endpoints.

In embodiments, the thermoplastic polymer may comprise an izod impact strength, notched, greater than or equal to 4.5 g/m2, greater than or equal to 5.0 g/m², or even greater than or equal to 5.5 g/m², measured at 23° C. In embodiments, the thermoplastic polymer may comprise an izod impact strength, notched, less than or equal to 8.0 g/m², less than or equal to 7.5 g/m², or even less than or equal to 7.0 g/m², measured at 23° C. In embodiments, the thermoplastic polymer may comprise an izod impact strength, notched, from 4.5 g/m² to 7.0 g/m², from 4.5 g/m² to 7.5 g/m², from 4.5 g/m² to 8.0 g/m², from 5.0 g/m² to 7.0 g/m², from 5.0 g/m² to 7.5 g/m², from 5.0 g/m² to 8.0 g/m², from 5.5 g/m² to 7.0 g/m², from 5.5 g/m² to 7.5 g/m², or even from 5.5 g/m² to 8.0 g/m², or any and all sub-ranges formed from any of these endpoints, measured at 23° C.

In embodiments, the thermoplastic polymer may comprise an izod impact strength, notched, greater than or equal to 2.5 g/m², greater than or equal to 3.0 g/m², or even greater than or equal to 3.5 g/m², measured at −20° C. In embodiments, the thermoplastic polymer may comprise an izod impact strength, notched, less than or equal to 6.0 g/m², less than or equal to 5.5 g/m², or even less than or equal to 5.0 g/m², measured at −20° C. In embodiments, the thermoplastic polymer may comprise an izod impact strength, notched, from 2.5 g/m² to 5.0 g/m², from 2.5 g/m² to 5.5 g/m², from 2.5 g/m² to 6.0 g/m², from 3.0 g/m² to 5.0 g/m², from 3.0 g/m² to 5.5 g/m², from 3.0 g/m² to 6.0 g/m², from 3.5 g/m² to 5.0 g/m², from 3.5 g/m² to 5.5 g/m², or even from 3.5 g/m² to 6.0 g/m², or any and all sub-ranges formed from any of these endpoints, measured at −20° C.

Suitable commercial embodiments of the thermoplastic polymer are available under the FORMOLENE brand from Formosa Plastics, such as polypropylene homopolymer grade 1102KR, the Sipolprene brand from SIPOL® SPA, such as 25170, the Rigidex brand from INEOS GROUP, such as 450-HP60the NHU brand from Zhejiang NHU Special Materials Co., Ltd., such as 1150C, or the Bondyram brand from Polyram, such as 1001.

### Filler

As described herein, using a filler comprising tungsten and barium sulfates produces a radiopaque polymer formulation having the desired dielectric strength before x-ray exposure, after x-ray exposure, or both and desired high absorption and low reflectivity while maintaining the mechanical properties of thermoplastic polymer.

A filler consisting of tungsten has high dielectric properties, but also has high reflective properties. A filler consisting of barium sulfate has high dielectric stability and absorption, but does not have the reflectivity required for x-ray applications. The presence and specific amounts of both tungsten and barium sulfate provide both desired dielectric strength before x-ray exposure, after x-ray exposure, or both and desired high absorption and low reflectivity for radiopaque polymer formulation. Tungsten provides the dielectric strength and the reflectivity necessary for x-ray applications. Barium sulfate provides the relatively high dielectric strength and absorption that may be desired in medical applications. The radiopaque polymer formulation comprising the filler comprising of tungsten and barium sulfate remains stable after high intensity of X-ray exposure, such as greater than or equal to 900 kGy (kilogray), while having desired absorption.

In embodiments, the filler may consist essentially of tungsten and barium sulfates. In embodiments, the filler may not include bismuth subcarbonate, bismuth trioxide, bismuth oxychloride, or combinations thereof. In embodiments, the filler may further comprise non-radiopaque fillers.

The filler comprises tungsten. Tungsten has reflection property because of its electrical shielding property being the dominant at high loading.

Various tungsten are considered suitable for the present filler of radiopaque polymer formulation. In embodiments, tungsten may be in an oxide form. In embodiments, tungsten may be in a powder form.

In embodiments, tungsten may have an average particle size of greater than or equal to 4 micrometer (μm), greater than or equal to 4.5 μm, or even greater than or equal to 5 μm. In embodiments, tungsten may have an average particle size of less than or equal to 16 μm, less than or equal to 15.5 μm, or even less than or equal to 15 μm. In embodiments, tungsten may have an average particle size of from 4 μm to 16 μm, from 4 μm to 15.5 μm, from 4 μm to 15 μm, from 4.5 um to 16 μm, from 4.5 μm to 15.5 μm, from 4.5 μm to 15 μm, from 5 μm to 16 μm, from 5 μm to 15.5 μm, or even from 5 μm to 15 μm, or any and all sub-ranges formed from any of these endpoints.

In embodiments, tungsten may be included in amounts greater than or equal to 25 wt %, greater than or equal to 28 wt %, or even greater than or equal to 30 wt %. In embodiments, the amount of tungsten in the radiopaque polymer formulation may be less than or equal to 45 wt %, less than or equal to 43 wt %, or even less than or equal to 42 wt %. In embodiments, the amount of thermoplastic polymer in the radiopaque polymer formulation may be from 25 wt % to 45 wt %, from 25 wt % to 43 wt %, from 25 wt % to 42 wt %, from 28 wt % to 45 wt %, from 28 wt % to 43 wt %, from 28 wt % to 42 wt %, from 30 wt % to 45 wt %, from 30 wt % to 43 wt %, or even from 30 wt % to 42 wt %, or any and all sub-ranges formed from any of these endpoints. When the amount of tungsten in the radiopaque polymer formulation is less than 25 wt %, the radiopaque polymer formulation may not have the reflectivity necessary for medical applications, such as X-ray applications. When the amount of tungsten in the radiopaque polymer formulation is greater than 45 wt %, the radiopaque polymer formulation may not have desired dielectric strength and absorption.

Suitable commercial embodiments of tungsten are available under the H.C. Starck brand from H.C. Starck GmbH, such as tungsten metal powder HC grade.

The filler comprises barium sulfate. Barium sulfate has attenuation because of it is insulative and has moderate thermal properties.

Various barium sulfate are considered suitable for the present filler of radiopaque polymer formulation. In embodiments, barium sulfate may be in a powder form.

In embodiments, barium sulfate may have a median size D50 of greater than or equal to 0.01 μm, greater than or equal to 0.05 μm, or even greater than or equal to 0.1 μm. In embodiments, barium sulfate may have a median size D50 of than or equal to 0.5 μm, less than or equal to 0.4 μm, or even less than or equal to 0.3 μm. In embodiments, barium sulfate may have a median size D50 of from 0.01 μm to 0.5 μm, from 0.01 μm to 0.4 μm, from 0.01 μm to 0.3 μm, from 0.05 μm to 0.5 μm, from 0.05 μm to 0.4 μm, from 0.05 μm to 0.3 μm, from 0.1 μm to 0.5 μm, from 0.1 μm to 0.4 μm, or even from 0.1 μm to 0.3 μm, or any and all sub-ranges formed from any of these endpoints.

In embodiments, the barium sulfate may be included in amounts greater than or equal to 35 wt %, greater than or equal to 38 wt %, or even greater than or equal to 40 wt %. In embodiments, the amount of barium sulfate in the radiopaque polymer formulation may be less than or equal to 55 wt %, less than or equal to 53 wt %, or even less than or equal to 51 wt %. In embodiments, the amount of thermoplastic polymer in the radiopaque polymer formulation may be from 35 wt % to 55 wt %, from 35 wt % to 53 wt %, from 35 wt % to 51 wt %, from 38 wt % to 55 wt %, from 38 wt % to 53 wt %, from 38 wt % to 51 wt %, from 40 wt % to 55 wt %, from 40 wt % to 53 wt %, or even from 40 wt % to 51 wt %, or any and all sub-ranges formed from any of these endpoints. When the amount of barium sulfate in the radiopaque polymer formulation is less than 35 wt %, the radiopaque polymer formulation may not have the desired absorption. When the amount of barium sulfate in the radiopaque polymer formulation is greater than 55 wt %, the radiopaque polymer formulation may have excessive dielectric strength before x-ray exposure, after x-ray exposure, or both.

Suitable commercial embodiments of barium sulfate are available under the BLANC FIXE brand from Blanc Fixe Micro or under BARIFLOR brand from Minerals Girona S.A., such as BARIFLOR 8410.

In embodiments, the amount of filler in the radiopaque polymer formulation may be greater than 55 wt %, greater than or equal to 57 wt %, greater than or equal to 60 wt %, greater than or equal to 62 wt %, or even greater than or equal to 65 wt %. In embodiments, the amount of filler in the radiopaque polymer formulation may be less than or equal to 95 wt %, less than or equal to 94 wt %, less than or equal to 93 wt %, less than or equal to 92 wt %, less than or equal to 91 wt %, or even less than or equal to 90 wt %. In embodiments, the amount of filler in the radiopaque polymer formulation may be from 55 wt % to 95 wt %, from 55 wt % to 94 wt %, from 55 wt % to 93 wt %, from 55 wt % to 92 wt %, from 55 wt % to 91 wt %, from 55 wt % to 90 wt %, from 57 wt % to 95 wt %, from 57 wt % to 94 wt %, from 57 wt % to 93 wt %, from 57 wt % to 92 wt %, from 57 wt % to 91 wt %, from 57 wt % to 90 wt %, from 60 wt % to 95 wt %, from 60 wt % to 94 wt %, from 60 wt % to 93 wt %, from 60 wt % to 92 wt %, from 60 wt % to 91 wt %, from 60 wt % to 90 wt %, from 62 wt % to 95 wt %, from 62 wt % to 94 wt %, from 62 wt % to 93 wt %, from 62 wt % to 92 wt %, from 62 wt % to 91 wt %, from 62 wt % to 90 wt %, from 65 wt % to 95 wt %, from 65 wt % to 94 wt %, from 65 wt % to 93 wt %, from 65 wt % to 92 wt %, from 65 wt % to 91 wt %, or even from 65 wt % to 90 wt %, or any and all sub-ranges formed from any of these endpoints.

### Radiopaque Polymer Formulation

As described herein, using a filler comprising tungsten and barium sulfates produces a radiopaque polymer formulation having desired dielectric strength before x-ray exposure, after x-ray exposure, or both and desired high absorption and low reflectivity while maintaining the mechanical properties of thermoplastic polymer.

In embodiments, the radiopaque polymer formulation may not include Manganese Zinc (Mn-Zn) Ferrite.

In embodiments, the radiopaque polymer formulation may have a dielectric strength under DC load greater than or equal to 3 kV/mm, greater than or equal to 3.5 kV/mm, or even greater than or equal to 4 kV/mm. In embodiments, the radiopaque polymer formulation may have a dielectric strength under DC load less than or equal to 6 kV/mm, less than or equal to 5.5 kV/mm, or even less than or equal to 5 kV/mm. In embodiments, the radiopaque polymer formulation may have a dielectric strength under DC load from 3 k V/mm to 6 kV/mm, from 3 kV/mm to 5.5 kV/mm, from 3 kV/mm to 5 kV/mm, from 3.5 kV/mm to 6 kV/mm, from 3.5 kV/mm to 5.5 kV/mm, from 3.5 kV/mm to 5 kV/mm, from 4 kV/mm to 6 kV/mm, from 4 kV/mm to 5.5 kV/mm, or even from 4 kV/mm to 5 kV/mm, or any and all sub-ranges formed from any of these endpoints.

In embodiments, the radiopaque polymer formulation may have a breakdown strength under DC load of greater than or equal to 10 kV/mm, greater than or equal to 12 kV/mm, or even greater than or equal to 15 kV/mm. In embodiments, the radiopaque polymer formulation may have a breakdown strength under DC load of less than or equal to 65 kV/mm, less than or equal to 60 kV/mm, or even less than or equal to 55 kV/mm. In embodiments, the radiopaque polymer formulation may have a breakdown strength under DC load of from 10 kV/mm to 65 kV/mm, from 10 kV/mm to 60 kV/mm, from 10 kV/mm to 55 kV/mm, from 12 kV/mm to 65 kV/mm, from 12 kV/mm to 60 kV/mm, from 12 kV/mm to 55 kV/mm, from 15 kV/mm to 65 kV/mm, from 15 kV/mm to 60 kV/mm, or even from 15 kV/mm to 55 kV/mm, or any and all sub-ranges formed from any of these endpoints.

In embodiments, the radiopaque polymer formulation may have a breakdown strength under DC load after X-ray exposure at greater than or equal to 900 kGy of greater than or equal to 5 kV/mm, greater than or equal to 6 kV/mm, or even greater than or equal to 8 kV/mm. In embodiments, the radiopaque polymer formulation may have a breakdown strength under DC load after X-ray exposure at greater than or equal to 900 kGy of less than or equal to 35 kV/mm, less than or equal to 32 kV/mm, or even less than or equal to 30 kV/mm. In embodiments, the radiopaque polymer formulation may have a breakdown strength under DC load after X-ray exposure at greater than or equal to 900 kGy of from 5 kV/mm to 35 kV/mm, from 5 kV/mm to 32 kV/mm, from 5 kV/mm to 30 kV/mm, from 6 kV/mm to 35 kV/mm, from 6 kV/mm to 32 kV/mm, from 6 kV/mm to 30 kV/mm, from 8 kV/mm to 35 kV/mm, from 8 kV/mm to 32 kV/mm, or even from 8 kV/mm to 30 kV/mm, or any and all sub-ranges formed from any of these endpoints.

In embodiments, the radiopaque polymer formulation may have an absorption of 1 mm lead (Pb) greater than or equal to 5 mm, greater than or equal to 5.5 mm, or even greater than or equal to 6 mm. In embodiments, the radiopaque polymer formulation may have an absorption of 1 mm Pb less than or equal to 10 mm, less than or equal to 8.5 mm, or even less than or equal to 7 mm. In embodiments, the radiopaque polymer formulation may have an absorption of 1 mm Pb from 5 mm to 10 mm, from 5 mm to 8.5 mm, from 5 mm to 7 mm, from 5.5 mm to 10 mm, from 5.5 mm to 8.5 mm, from 5.5 mm to 7 mm, from 6 mm to 10 mm, from 6 mm to 8.5 mm, or even from 6 mm to 7 mm, or any and all sub-ranges formed from any of these endpoints.

In embodiments, the radiopaque polymer formulation may have a charpy impact strength, notched, greater than or equal to 1.3 kJ/m², greater than or equal to 1.4 kJ/m², or even greater than or equal to 1.5 kJ/m². In embodiments, the radiopaque polymer formulation may have a charpy impact strength, notched, less than or equal to 6.5 kJ/m², less than or equal to 6.2 kJ/m², or even less than or equal to 6.0 KJ/m². In embodiments, the radiopaque polymer formulation may have a charpy impact strength, notched, from 1.3 kJ/m² to 6.5 kJ/m², from 1.3 kJ/m² to 6.2 KJ/m², from 1.3 kJ/m² to 6.0 KJ/m², from 1.4 kJ/m² to 6.5 kJ/m², from 1.4 kJ/m² to 6.2 kJ/m², from 1.4 KJ/m² to 6.0 KJ/m², from 1.5 kJ/m² to 6.5 kJ/m², from 1.5 kJ/m² to 6.2 kJ/m², or even from 1.5 kJ/m² to 6.0 KJ/m², or any and all sub-ranges formed from any of these endpoints

In embodiments, the radiopaque polymer formulation may have a charpy impact strength, unnotched, greater than or equal to 1.0 kJ/m², greater than or equal to 1.5 kJ/m², or even greater than or equal to 2.0 KJ/m². In embodiments, the radiopaque polymer formulation may have a charpy impact strength, unnotched, less than or equal to 30.0 KJ/m², less than or equal to 28.0 kJ/m², or even less than or equal to 26.0 KJ/m². In embodiments, the radiopaque polymer formulation may have a charpy impact strength, unnotched, from 1.0 KJ/m² to 30.0 KJ/m², from 1.0 KJ/m² to 28.0 KJ/m², from 1.0 KJ/m² to 26.0 KJ/m², from 1.5 kJ/m² to 30.0 KJ/m², from 1.5 kJ/m² to 28.0 KJ/m², from 1.5 kJ/m² to 26.0 KJ/m², from 2.0 KJ/m² to 30.0 KJ/m², from 2.0 KJ/m² to 28.0 kJ/m², or even from 2.0 KJ/m² to 26.0 KJ/m², or any and all sub-ranges formed from any of these endpoints

In embodiments, the radiopaque polymer formulation may have a tensile strength at break greater than or equal to 5 MPa, greater than or equal to 6 MPa, or even greater than or equal to 7 MPa. In embodiments, the radiopaque polymer formulation may have a tensile strength at break less than or equal to 45 MPa, less than or equal to 42 MPa, or even less than or equal to 40 MPa. In embodiments, the radiopaque polymer formulation may have a tensile strength at break from 5 MPa to 45 MPa, from 5 MPa to 42 MPa, from 5 MPa to 40 MPa, from 6 MPa to 45 MPa, from 6 MPa to 42 MPa, from 6 MPa to 40 MPa, from 7 MPa to 45 MPa, from 7 MPa to 42 MPa, or even from 7 MPa to 40 MPa, or any and all sub-ranges formed from any of these endpoints.

In embodiments, the radiopaque polymer formulation may have a tensile elongation at break greater than or equal to 0.15%, greater than or equal to 0.2%, or even greater than or equal to 0.25%. In embodiments, the radiopaque polymer formulation may have a tensile elongation at break less than or equal to 5.5%, less than or equal to 5.0% or even less than or equal to 4.5%. In embodiments, the radiopaque polymer formulation may have a tensile elongation at break from 0.15% to 5.5%, from 0.15% to 5.0%, from 0.15% to 4.5%, from 0.2% to 5.5%, from 0.2% to 5.0%, from 0.2% to 4.5%, from 0.25% to 5.5%, from 0.25% to 5.0%, or even from 0.25% to 4.5%, or any and all sub-ranges formed from any of these endpoints.

The radiopaque polymer formulations described herein comprising thermoplastic polymer, and a filler comprising tungsten and barium sulfate, exhibits both desired dielectric strength before x-ray exposure, after x-ray exposure, or both and desired absorption while maintaining the mechanical properties of thermoplastic polymer.

### Antioxidant

In embodiments, the radiopaque polymer formulations described herein may further comprise antioxidant. In embodiments, the antioxidants may be a primary phenolic, a hindered amine light stabilizer, or combinations thereof.

In embodiments, the antioxidant may comprise tetrakis [methylene-3-(3,5-di-tert-butyl-4-hydroxyphenyl-propionate)] methane. In embodiments, the antioxidant may comprise a phenolic antioxidant.

In embodiments, the amount of antioxidant in the radiopaque polymer formulation may be greater than or equal to 0.001 wt %, greater than or equal to 0.005 wt %, or even greater than or equal to 0.01 wt %. In embodiments, the amount of antioxidant in the radiopaque polymer formulation may be less than or equal to 2 wt %, less than or equal to 1.5 wt %, or even less than or equal to 1 wt %. In embodiments, the amount of antioxidant in the radiopaque polymer formulation may be from 0.001 wt % to 2 wt %, from 0.001 wt % to 1.5 wt %, from 0.001 wt % to 1 wt %, from 0.005 wt % to 2 wt %, from 0.005 wt % to 1.5 wt %, from 0.005 wt % to 1 wt %, from 0.01 wt % to 2 wt %, from 0.01 wt % to 1.5 wt %, or even from 0.01 wt % to 1 wt %, or any and all sub-ranges formed from any of these endpoints.

Suitable commercial embodiments of antioxidants are available under the IRGANOX brand from BASF, such as grade 1010.

### Fatty Acid Amide

In embodiments, the radiopaque polymer formulations described herein may further comprise fatty acid amide to improve processing of radiopaque polymer formation in extrusion as well injection molding.

In embodiments, the fatty acid amide may comprise ethylene bis-stearamide (EBS).

In embodiments, the amount of fatty acid amide in the radiopaque polymer formulation may be greater than or equal to 0.001 wt %, greater than or equal to 0.005 wt %, or even greater than or equal to 0.01 wt %. In embodiments, the amount of fatty acid amide in the radiopaque polymer formulation may be less than or equal to 2 wt %, less than or equal to 1.5 wt %, or even less than or equal to 1 wt %. In embodiments, the amount of fatty acid amide in the radiopaque polymer formulation may be from 0.001 wt % to 2 wt %, from 0.001 wt % to 1.5 wt %, from 0.001 wt % to 1 wt %, from 0.005 wt % to 2 wt %, from 0.005 wt % to 1.5 wt %, from 0.005 wt % to 1 wt %, from 0.01 wt % to 2 wt %, from 0.01 wt % to 1.5 wt %, or even from 0.01 wt % to 1 wt %, or any and all sub-ranges formed from any of these endpoints.

Suitable commercial embodiments of the fatty acid amide are available under the Crodamide brand, such as EBS, from Croda Industrial Chemicals.

### Thioester

In embodiments, the radiopaque polymer formulations described herein may further

comprise thioester to improve processing and stability of radiopaque polymer formation in extrusion as well injection molding.

In embodiments, the thioester may comprise dialkyl ester of thiodipropionic acid.

In embodiments, the amount of thioester in the radiopaque polymer formulation may be greater than or equal to 0.001 wt %, greater than or equal to 0.005 wt %, or even greater than or equal to 0.01 wt %. In embodiments, the amount of thioester in the radiopaque polymer formulation may be less than or equal to 2 wt %, less than or equal to 1.5 wt %, or even less than or equal to 1 wt %. In embodiments, the amount of thioester in the radiopaque polymer formulation may be from 0.001 wt % to 2 wt %, from 0.001 wt % to 1.5 wt %, from 0.001 wt % to 1 wt %, from 0.005 wt % to 2 wt %, from 0.005 wt % to 1.5 wt %, from 0.005 wt % to 1 wt %, from 0.01 wt % to 2 wt %, from 0.01 wt % to 1.5 wt %, or even from 0.01 wt % to 1 wt %, or any and all sub-ranges formed from any of these endpoints.

Suitable commercial embodiments of the thioester are available under the IRGANOX brand, such as PS 880 FL, from BASF.

### Additives

In embodiments, the radiopaque polymer formulation may further comprise an additive. In embodiments, the additive may comprise adhesion promoters; biocides; anti-fogging agents; anti-static agents; blowing and foaming agents; bonding agents and bonding polymers; polar copolymers (e.g., ethylene-vinyl acetate (EVA), ethylene butyl acrylate (EBA), or ethyl methacrylate (EMA)); dispersants; flame retardants and smoke suppressants; mineral fillers; initiators; lubricants; micas; pigments, colorants, and dyes; processing aids; release agents; silanes, titanates, and zirconates; slip and anti-blocking agents; ultraviolet light stabilizer; viscosity regulators; waxes; or a combination thereof. In embodiments, the additive may comprise calcium stearates, didodecyl-3,3′-thiodipropionate, or both.

In embodiments, the amount of additives in the radiopaque polymer formulation may be greater than or equal to 0.001 wt %, greater than or equal to 0.005 wt %, or even greater than or equal to 0.01 wt %. In embodiments, the amount of additives in the radiopaque polymer formulation may be less than or equal to 2 wt %, less than or equal to 1.5 wt %, or even less than or equal to 1 wt %. In embodiments, the amount of additives in the radiopaque polymer formulation may be from 0.001 wt % to 2 wt %, from 0.001 wt % to 1.5 wt %, from 0.001 wt % to 1 wt %, from 0.005 wt % to 2 wt %, from 0.005 wt % to 1.5 wt %, from 0.005 wt % to 1 wt %, from 0.01 wt % to 2 wt %, from 0.01 wt % to 1.5 wt %, or even from 0.01 wt % to 1 wt %, or any and all sub-ranges formed from any of these endpoints.

### Process

In embodiments, the radiopaque polymer formulation described herein may be made with a batch process or continuous process.

In embodiments, the components of the radiopaque polymer formulation, including the thermoplastic polymer, and the filler comprising tungsten and barium sulfate, may be added to an extruder (e.g., ZSK 26MC-Coperion Extruder) and blended. In embodiments, the blending (e.g., in the barrel of the extruder) may be carried out at a temperature from 80° C. to 350° C.

Blending (also known as compounding) devices are well known to those skilled in the art and generally include feed means, especially at least one hopper for pulverulent materials and/or at least one injection pump for liquid materials; high-shear blending means, for example a co-rotating or counter-rotating twin-screw extruder, usually comprising a feed screw placed in a heated barrel (or tube); an output head, which gives the extrudate its shape; and means for cooling the extrudate, either by air cooling or by circulation of water. The extrudate is generally in the form of rods continuously exiting the device and able to be cut or formed into granules. However, other forms may be obtained by fitting a die of desired shape on the output die. For example, in embodiments, the process may comprise profile extrusion including forcing the extrudate through a die cut into the linear shape of the desired finished radiopaque polymer formulation (e.g., channel or tube).

It will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these aspects.

What is claimed is:

1. A radiopaque polymer formulation comprising:
thermoplastic polymer; and
a filler comprising:
25 weight percent (wt %) to 45 wt % of tungsten, based on the total amount of the radiopaque polymer formulation; and
35 wt % to 55 wt % of barium sulfate, based on the total amount of the radiopaque polymer formulation.

2. The radiopaque polymer formulation of claim 1, wherein the radiopaque polymer formulation has dielectric strength under DC load of from 3 kV/mm to 6 kV/mm, or from 4 kV/mm to 5 kV/mm.

3. The radiopaque polymer formulation of claim 1, wherein the radiopaque polymer formulation has absorption of 1 millimeter (mm) lead (Pb) from 5 to 10 mm, or from 6 to 7 mm.

4. The radiopaque polymer formulation of claim 1, wherein the radiopaque polymer formulation comprises 7 wt % to 35 wt %, 9 wt % to 32 wt %, or 11 wt % to 30 wt % of the thermoplastic polymer, based on the total amount of the radiopaque polymer formulation.

5. The radiopaque polymer formulation of claim 1, wherein the thermoplastic polymer comprises thermoplastic polyolefin, thermoplastic elastomer, or both.

6. The radiopaque polymer formulation of claim 1, wherein the thermoplastic polymer comprises polypropylene (PP), polyphenylene sulfide (PPS), polybutylene terephthalate (PBT), thermoplastic polyester elastomers (TPC), or a combination thereof.

7. The radiopaque polymer formulation of claim 1, wherein the radiopaque polymer formulation comprises 28 wt % to 43 wt % of tungsten, or 30 wt % to 42 wt % of tungsten, based on the total amount of the radiopaque polymer formulation.

8. The radiopaque polymer formulation of claim 1, wherein the radiopaque polymer formulation comprises 38 wt % to 53 wt % of barium sulfate, or 40 wt % to 51 wt % of barium sulfate, based on the total amount of the radiopaque polymer formulation.

9. The radiopaque polymer formulation of claim 1, wherein the radiopaque polymer formulation further comprises 0.01 wt % to 1 wt % of an antioxidant, based on the total amount of the radiopaque polymer formulation.

10. The radiopaque polymer formulation of claim 1, wherein the radiopaque polymer formulation further comprises 0.01 wt % to 1 wt % of fatty acid amide, based on the total amount of the radiopaque polymer formulation.

11. The radiopaque polymer formulation of claim 10, wherein the fatty acid amide comprises ethylene bis-steramide (EBS).

12. The radiopaque polymer formulation of claim 1, wherein the radiopaque polymer formulation further comprises 0.01 wt % to 1 wt % of thioester, based on the total amount of the radiopaque polymer formulation.

13. The radiopaque polymer formulation of claim 12, wherein the thioester comprises dialkyl ester of thiodipropionic acid.

14. The radiopaque polymer formulation of claim 1, wherein the formulation further comprises 0.01 wt % to 1 wt % of an additive, based on the total amount of the radiopaque polymer formulation.

15. A medical device comprising the radioactive polymer formulation of claim 1.

16. The medical device of claim 15, wherein the medical device is an X-ray shielding component.
